(19) United States
(12) Patent Application Publication
(10) Pub. No.: US 2023/0177205 A1
(43) Pub. Date: Jun. 8, 2023

(54) SYSTEMS AND METHODS TO FACILITATE MANAGEMENT OF ONLINE SUBJECT INFORMATION

(71) Applicant: Solsten, Inc., St. Paul, MN (US)

(72) Inventors: Joseph Jack Schaeppi, Maple Grove, MN (US); Aliaksandr Pliutau, Berlin (DE)

(21) Appl. No.: 17/545,866

(22) Filed: Dec. 8, 2021

(51) Int. Cl. G06F 21/62; G16H 20/70

(52) U.S. Cl. CPC G06F 21/6245; G16H 20/70

(57) ABSTRACT

Systems and methods to facilitate management of online subject information are disclosed. Exemplary implementations may: store, in electronic storage, subject information for subjects; receive, from an online platform, a request for the psychological profile related to the given subject; authenticate, responsive to the request, the given subject by obtaining and analyzing authentication information for the given subject; determine, responsive to authenticating the given subject, permission information for the given subject that indicates permissions for transmitting the psychological profile of the given subject to one or more of the online platforms including the online platform; transmit, based on the permission information, the psychological profile to the online platform; receive, from the online platform, additional usage information related to the given subject and the online platform; and supplement the subject information for the given subject stored in the electronic storage with the additional usage information.

### FIELD OF THE DISCLOSURE

The present disclosure relates to systems and methods to facilitate management of online subject information.

### BACKGROUND

Online platforms may be dynamic to tailor their content to individual subjects. Current techniques of tailoring the content to the individual subjects may include collecting personal identification information, tracing interactions of individual subjects, and/or other techniques that may invade subject privacy as the subject privacy in online settings is becoming more and more important to the subjects.

### SUMMARY

One aspect of the present disclosure relates to subjects managing their online subject information that characterizes their online individualities without personally identifying them, i.e., what makes them unique from other subjects. Individual subjects may be authenticated to verify the subjects and identify their corresponding subject information. The subjects may provide permission for access or use of their subject information by online platforms. Based on the permission, the subject information, or part thereof, may be transmitted to one or more online platforms that requested it. As such, the subjects may manage who or what entities may access or use their subject information, how they utilize their subject information, when they have access, and/or other management. Thus, entities (i.e., companies or people) may not have sole control over the subjects subject information online, instead of the subjects themselves.

One aspect of the present disclosure relates to a system configured to facilitate management of online subject information. The system may include one or more hardware processors configured by machine-readable instructions, electronic storage, and/or other elements. Machine-readable instructions may include one or more instruction components. The instruction components may include computer program components. The instruction components may include one or more of request receiving component, subject authentication component, permission component, profile transmittal component, supplementation component, and/or other instruction components.

The electronic storage may be configured to store subject information for subjects. The subject information may represent online individualities of the subjects. The subject information for a given subject may include usage information, stated information, a psychological profile, and/or other information. The usage information may characterize usage of applications within digital environments provided by online platforms. The stated information may include answers to questions presented to the given subject that are related to psychological attributes. The psychological profile may include psychological parameter values to psychological parameters derived from the usage information and the stated information.

The request receiving component may be configured to receive, from an online platform, a request for the psychological profile related to the given subject.

The subject authentication component may be configured to authenticate, responsive to the request, the given subject by obtaining and analyzing authentication information for the given subject.

The permission component may be configured to determine, responsive to authenticating the given subject, permission information for the given subject that indicates permissions for transmitting the psychological profile of the given subject to one or more of the online platforms including the online platform.

The profile transmittal component may be configured to transmit, based on the permission information, the psychological profile to the online platform.

The supplementation component may be configured to receive, from the online platform, additional usage information related to the given subject and the online platform. The supplementation component may be configured to supplement the subject information for the given subject stored in the electronic storage with the additional usage information.

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates a system configured to facilitate management of online subject information, in accordance with one or more implementations.

FIG. 2 illustrates a method to facilitate management of online subject information, in accordance with one or more implementations.

FIG. 3 may illustrate an example implementation, in accordance with one or more implementations.

### DETAILED DESCRIPTION

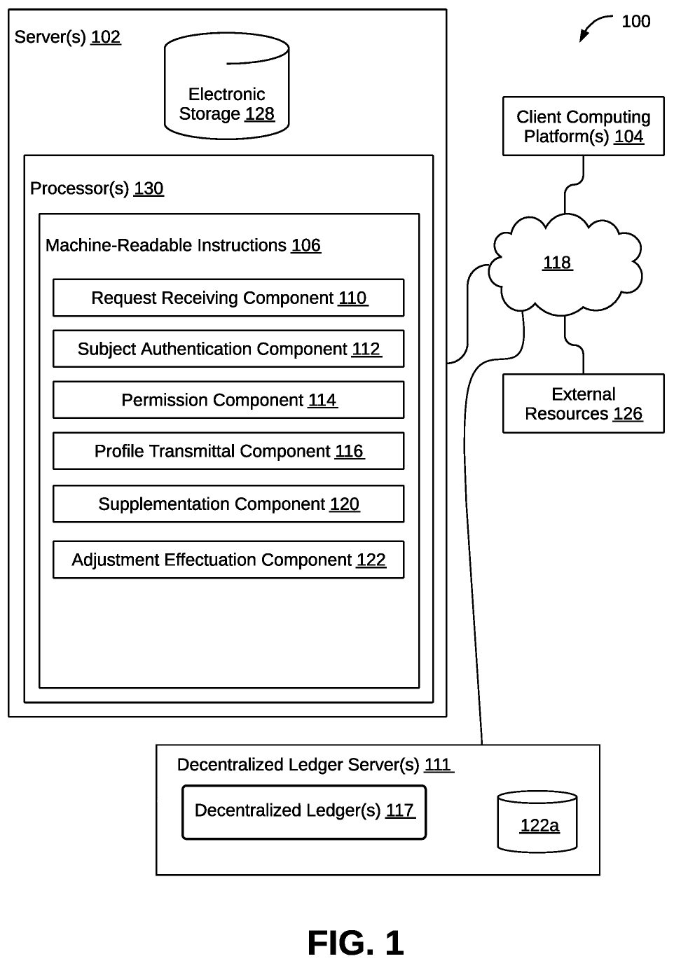

FIG. 1 illustrates a system 100 configured to facilitate management of online subject information, in accordance with one or more implementations. In some implementations, system 100 may include one or more servers 102, client computing platforms 104, decentralized ledger server(s) 111. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Subjects may access system 100 via client computing platform(s) 104.

Server(s) 102 may be configured by machine-readable instructions 106. Machine-readable instructions 106 may include one or more instruction components. The instruction components may include computer program components. The instruction components may include one or more of request receiving component 110, subject authentication component 112, permission component 114, profile transmittal component 116, supplementation component 120, adjustment effectuation component 122, and/or other instruction components.

Electronic storage 128 may be configured to store subject information for subjects and/or other information. The subjects may be users of online platforms and/or digital environments provided by the online platforms. The subject information may represent online individualities of the subjects. That is, individual subject information may be individual or unique to individual ones of the subjects. The subject information for a given subject may include usage information, stated information, interaction information, a psychological profile, and/or other information.

The usage information may characterize usage of applications within digital environments, online platforms, and/or other providers. The digital environments may include one or more applications that are usable by subjects (e.g., digital application environments), a simulated space that is accessible by subjects via clients (e.g., client computing platform(s) 104) that present views of a virtual space to a subject, content, and/or other digital environment elements.

The simulated space or simulated spaces provided by the individual applications may have a topography, express ongoing real-time interaction by one or more subjects, and/or include one or more objects positioned within the topography that are capable of locomotion within the topography. In some instances, the topography may be a 2-dimensional topography. In other instances, the topography may be a 3-dimensional topography. The topography may include dimensions of the space, and/or surface features of a surface or objects that are “native” to the space. In some instances, the topography may describe a surface (e.g., a ground surface) that runs through at least a substantial section of the space. In some instances, the topography may describe a volume with one or more bodies positioned therein (e.g., a simulation of gravity-deprived space with one or more celestial bodies positioned therein). The instance executed by the computer components may be synchronous, asynchronous, and/or semi-synchronous.

By way of non-limiting example, the content may include one or more of a character, a game, a game asset, a recommendation, a suggestion, a promotion, and/or other content. The character may refer to an object (or group of objects) present in a virtual space that corresponds to an individual subject (e.g., an avatar) and/or are controlled by the subject. In some implementations, the character may not correspond to an individual subject but rather provide information (e.g., the recommendation, the suggestion) to the subject. The game asset may include a virtual item, a virtual resource (e.g., weapon, tool), of in-game powers, in-game skills, in-game technologies, and/or other game assets. The recommendation may include a particular selection and/or action that the subject is advised to select and/or do. The suggestion may include particular ideas, plans, and/or strategies for the subject to consider executing, following, and/or is determined they will enjoy. The promotion may include discount codes, coupons, bonuses, and/or other promotions of the virtual items, products, and/or services that the subject may utilize. By way of non-limiting example, products and/or services may relate to beauty (e.g., skincare, makeup), home improvement, decoration, clothing, accessories, technology, kitchen, and/or other categories.

The above description of the manner in which views of the virtual space are provided is not intended to be limiting. The virtual space may be expressed in a more limited, or richer, manner. For example, views determined for the virtual space may be selected from a limited set of graphics depicting an event in a given place within the virtual space. The views may include additional content (e.g., text, audio, pre-stored video content, and/or other content) that describes particulars of the current state of the place, beyond the relatively generic graphics. For example, a view may include a generic battle graphic with a textual description of the opponents to be confronted. Other expressions of individual places within the virtual space are contemplated.

In some implementations, the digital environments may be provided by computer components in server(s) 102, client computing platforms 104, servers associated with online platforms that host the individual applications, or other sources. As such, the digital environments may include one or more of the applications. In some implementations, the digital environments may be provided by the online platforms and accessible by the subjects via web browsers (e.g., suite of applications, online games). In some implementations, the individual applications may be provided by the online platforms and accessible by the subjects via web browsers (i.e., web version of a desktop game).

The applications, and application types thereof, may include game applications, educational applications, reading application, music applications, social networking applications, entertainment applications, fitness applications, business applications, shopping applications, food & drink applications, among other applications.

In some implementations, the usage information may be obtained from individual ones of the applications installed on client computing platforms 104. In some implementations, the usage information may be obtained from the operating systems that aggregate the usage information from the individual applications of client computing platforms 104. In some implementations, the usage information may be obtained from a recordation application installed on client computing platforms 104 that aggregates the application usage information from the individual applications. In some implementations, the usage information may be obtained from the online platforms (e.g., that host the applications, online games, etc.), and/or other obtainment techniques.

The usage information may include values to screen time, battery usage, Internet usage, location usage, times of installations of the individual applications, the application types of installations of the individual applications, costs of the installations, times of initiations of the individual applications, times of terminations of the individual applications, amount of the notifications, notification types of the notifications, cross-application information usage, times of in-application purchases and in-application sales, item type of the in-application purchases, the item type of the in-application sales, content types of content interacted with, interaction types of interactions, the application types of the applications initiated, and/or other usage parameters. The term “in-application” as used herein may include “in-platform” as the usage information may refer to usage of the online platforms and/or the applications.

The stated information for the given subject and other ones of the subjects, may include individual sets of answers to questions presented to the individual subjects that are related to psychological attributes. The individual sets of answers may be provided by individual ones of the subjects. In some implementations, the stated information may be obtained as described in co-pending U.S. Application Serial No. 16/854,660 entitled “SYSTEMS AND METHODS FOR ADAPTING USER EXPERIENCE IN A DIGITAL EXPERIENCE BASED ON PSYCHOLOGICAL ATTRIBUTES OF INDIVIDUAL USERS”, Attorney Docket No. 01TT-064001, the disclosure of which is incorporated by reference in its entirety herein.

The interaction information may characterize the content engaged with by individual subjects, engagement by the individual subjects with the content, and/or other interaction information. The content engaged with by the individual subjects may be related to the online platforms that provide the content. That is, for example, the content provided by an online gaming platform may relate to online games (e.g., virtual goods, virtual mini-games, etc.). In some implementations, the content engaged with by the individual subjects may not be related to the online platforms that provide the content.

The engagement by the individual subjects may define behavior patterns of the individual subjects with or based on the content. The behavior patterns of the individual subjects may include individual actions, sets of actions, ordered sets of actions, time spent by the individual subjects engaging with the content and/or the online platforms, spending patterns of the subjects, completed tasks by the individual subjects, uncompletion tasks by the individual subjects, failure of tasks by the individual subjects, and/or other behavior patterns. In some implementations, the behavior patterns may include multiple of the individual actions, the sets of actions, and the ordered set of actions. The actions may include one or more of a purchase based on the content, a sale, a trade, a donation, a subject selection of the content, gameplay (e.g., mini-game, battle, competition, etc.) based on the content, communication of the individual subjects with other particular subjects, completion of tasks by the subjects, frequent interaction with the content, formation of alliances by the subjects, and/or other actions. The spending patterns may indicate an amount of currency (e.g., real-world money, virtual money, points, etc.) spend, an amount of currency earned, an amount of currency donated, and/or other indications.

In some implementations, the interactions of the subjects with the digital environments and/or the behavior patterns of the subjects may be correlated with psychological attributes as described in U.S. Application Serial No. 16/894,522 entitled “SYSTEMS AND METHODS TO CORRELATE USER BEHAVIOR PATTERNS WITHIN AN ONLINE GAME WITH PSYCHOLOGICAL ATTRIBUTES OF USERS” Attorney Docket No. 01TT-064002, the disclosure of which is incorporated by reference in its entirety herein.

The psychological profile for the given subject may include psychological parameter values to psychological parameters derived from the usage information for the given subject, the stated information for the given subject, and/or other information. Similarly, psychological profiles for the individual subjects may include sets of psychological parameter values to the psychological parameters for the individual subjects.

The psychological parameter values may characterize, by way of non-limiting example, achievement motivation, motivation, personality inventory, cultural values, competitiveness, positive and negative affect before, during, and/or after engagement with the digital experience (i.e., emotions), communication style, personal values, daily routines/activities, life/gaming pain points, life/gaming hopes and aspirations, wellbeing, user experience, gaming/experience using time, subscription behavior, affinity information, personality, emotional style, goal orientation, goal commitment, ego and task orientation, relatedness, sense of community, social influence, social identity, group identification, we-identity, quality of life, satisfaction with life, work-related quality of life, mindfulness, happiness, emotional intelligence, self-awareness/internal awareness, external awareness, connectedness to nature, social connectedness, social bonding, perceived stress, depression, anxiety, decision-making style, thinking style, critical thinking, cognitive approach to learning, learning style, attributional style, internality-externality, stability-instability, global-specific, creativity, curiosity, playfulness, exploration, mental strength, grit, flourishing, gratitude, inspiration, spirituality, hedonism, materialism/material values, perceptions, sentiments, and/or other psychological parameters.

Achievement motivation may include compensatory effort, competitiveness, confidence in success, dominance, eagerness to learn, engagement, fearlessness, flexibility, flow, goal setting, independence, internality, persistence, preference in difficult tasks, pride in productivity, self-control, status orientation, ambition, self-assurance, and/or other psychological parameters. Motivation may include mastery, purpose, autonomy, and/or other psychological parameters.

Personality inventory may include neuroticism, openness, conscientiousness, extraversion, and agreeableness and/or other psychological parameters. Neuroticism may include anxiety, impulsiveness, vulnerability, and/or other psychological parameters. Openness may include fantasy, feelings/empathy, action, and/or other psychological parameters. Conscientiousness may include achievement striving, competence, self-discipline, and/or other psychological parameters. Extraversion may include warmth assertiveness, activity, and/or other psychological parameters. Agreeableness may include trust, altruism, modesty, and/or other psychological parameters.

Cultural values may include individualism, indulgence, long term orientation, masculinity, power distance, uncertainty avoidance, and/or other psychological parameters. Competitiveness may include avoidant, collaborative, competitive affectivity, dependent, dominant, general competitiveness, independent, personal enhancement, and/or other psychological parameters.

Positive and negative affect before, during, and/or after engaging in the digital experience may include hostility, joviality, negative emotions, positive emotions, sadness, self-assurance, and/or other psychological parameters. Communication style may include feeler, intuitor, sensor, thinker, and/or other psychological parameters.

Wellbeing may include social wellbeing, psychological wellbeing, physical wellbeing, physical activity, sleep, bounded reciprocity, resilience grit, and/or other psychological parameters.

Personality may include anger, hostility, depression, self-conscientiousness, excitement-seeking, positive emotions, gregariousness, ideas, values, aesthetics, tender-mindedness, straightforwardness, compliance, deliberation, order, dutifulness, and/or other psychological parameters.

Emotional style may include resilience, outlook, social intuition, self-awareness, sensitivity to context, attention, and/or other psychological parameters.

Goal orientation may include mastery approach/learning goal orientation, performance approach/performance goal orientation, performance avoid/avoidance goal orientation, and/or other psychological parameters.

Work-related quality of life may include structure, boundaries, focus, efficiency, information provision, communication, psychological support, stress at/from work, psychological safety, connectedness with team, motivation to work, adaptability, job/career satisfaction, control at work, home-work interface, general wellbeing, working conditions, and/or other psychological parameters.

Mindfulness may include observing, describing, acting with awareness, non-judgment, non-reactivity, and/or other psychological parameters.

Emotional intelligence may include emotion perception, emotion expression, emotion management, emotion regulation, impulse control, relationships, stress management, and/or other psychological parameters.

Social connectedness may include social connectedness, loneliness, membership self-esteem, private self-esteem, public self-esteem identity self-esteem, interdependent self, independent self, social avoidance, social distress, and/or other psychological parameters.

Decision-making style may include respected, confident, spontaneous, dependent, vigilant, avoidant, brooding, intuitive, anxious, and/or other psychological parameters.

Thinking style may include intuitive, experiential, analytical, rational, and/or other psychological parameters.

Cognitive approaches to learning may include avoidant, participative, competitive, collaborative, dependent, independent, and/or other psychological parameters.

Learning style may include visual (spatial), aural (auditory-musical), verbal (linguistic), physical (kinesthetic), logical (mathematical), social (interpersonal), solitary (intrapersonal), and/or other psychological parameters.

Mental strength may include tenacity, confidence, optimism, adaptability, self-awareness, reliability, responsibility, well-being, and/or other psychological parameters.

Flourishing may include positive emotion, engagement, relationships, meaning, accomplishment, health, loneliness, and/or other psychological parameters.

By way of non-limiting example, the psychological parameter values of the psychological parameters may be a number score on a predetermined range unique to each psychological parameter, a letter score, and/or other type of value than may characterize a particular subject as whole.

In some implementations, the psychological profiles and/or the subject information of the subjects without identifying the subjects personally with the personal identification information may be determined and stored to electronic storage 128 as described in U.S. Application Serial No. 17/157,712 entitled “SYSTEMS AND METHODS TO DETERMINE CONTENT TO PRESENT BASED ON INTERACTION INFORMATION OF A GIVEN USER” Attorney Docket No. 01 TT-064004, the disclosure of which is incorporated by reference in its entirety herein.

In some implementations, the subject information for the subjects may be correlated with subject record identifiers. The subject record identifiers may be unique to the subject information for the individual subjects. In some implementations, the subject information may be stored and identifiable in electronic storage 128 by its subject record identifier. The subject record identifiers may be associated with non-fungible tokens. A decentralized ledger, such as decentralized ledger(s) 117, may indicate ownership of the non-fungible tokens by way of recordation of the subject record identifiers.

Decentralized ledger server(s) 111 may be used to implement one or more decentralized ledger(s) 117. Decentralized ledger server(s) 111 may include electronic storage 122a similar to electronic storage 128. In some implementations, decentralized ledger(s) 117 may be maintained by distributed computing platforms (not shown in FIG. 1). In some implementations, a distributed computing platform may be implemented by a set of client computing platforms and/or servers (including, for example, one or more decentralized ledger server(s) 111 and/or client computing platform(s) 104). The distributed computing platform may support a virtual machine (not shown in FIG. 1). The distributed computing platform and/or the virtual machine may form a runtime environment for smart contracts and/or other executable code. A distributed computing platform may include electronic storage configured to store part or all of decentralized ledger(s) 117. The smart contracts may be stored on decentralized ledger(s) 117 and/or another decentralized ledger. In some implementations, the distributed computing platform may be the EOSIO platform. In some implementations, the distributed computing platform may be similar to or based on the EOSIO platform. In some implementations, the distributed computing platform may be Ethereum. In some implementations, the distributed computing platform may be similar to or based on Ethereum. In some implementations, the virtual machine may be a decentralized virtual machine.

In some implementations, at least one of the decentralized ledger(s) 117 implemented by decentralized ledger server(s) 111 is a private permissioned decentralized ledger. The private permissioned decentralized ledger may be configured to record information. The recorded information may pertain to one or more non-fungible tokens recorded on decentralized ledger(s) 117. The recorded information may include ownership of the non-fungible tokens and/or other assets. In some implementations, the ownership of the non-fungible tokens and/or other assets may be indicated by digital address, that correspond to digital wallets of the subjects, recorded and/or tracked by decentralized ledger(s) 117. Ownership rights and/or other rights may be modified. In some implementations, a non-fungible token may be removed from one decentralized ledger and added or recorded on another decentralized ledger. In some implementations, at least one of the decentralized ledger(s) 117 implemented by decentralized ledger server(s) 111 is a public decentralized ledger. The public decentralized ledger may be configured to be part of either EOSIO mainnet, Ethereum mainnet, Ethereum 1.5, Ethereum 2.0, a derivative of Ethereum 2.0 that is configured to perform transactions of Ether (ETH) between accounts, or a derivative of EOSIO that is configured to perform transactions of EOS between different accounts.

Elements of decentralized ledger(s) 117 ledger may be grouped together in units that are referred to as blocks and/or nodes. For example, an individual block may include one or more assets (e.g., non-fungible tokens) and/or one or more transactions. For example, an individual block may be linked to one or more other individual blocks. Individual blocks may be linked or chained together to form a structure of blocks and/or a hierarchy of blocks, such as, e.g., a chain of blocks. An individual block may include one or more assets (e.g., non-fungible tokens), one or more transactions, and/or other information.

In some implementations, an individual decentralized ledger server(s) 111 may be dedicated to a particular node of a decentralized ledger(s) 117. Typically, different nodes are included in (or implemented by, or hosted by) different servers or different computer systems to increase the safety and security of transactions on a decentralized ledger and/or blockchain. The consensus protocol used for a particular blockchain will be harder to falsify or circumvent when the different nodes are in different geographical locations, on different types of computing platforms, and/or otherwise distributed and diverse.

In some implementations, one or more decentralized ledger(s) 117 implemented by decentralized ledger server(s) 111 may be publicly accessible. In some implementations, one or more decentralized ledger(s) 117 implemented by decentralized ledger server(s) 111 may be private and/or permissioned. In some implementations, one or more decentralized ledger(s) 117 implemented by decentralized ledger server(s) 111 may be append-only. In some implementations, existing blocks and/or nodes of one or more decentralized ledger(s) 117 implemented by decentralized ledger server(s) 111 can substantially not be altered or deleted, unless multiple copies are altered. This is unlikely to happen provided that the multiple copies stored on different computing platforms, e.g., in different geographical locations, in accordance with the relevant consensus protocol(s). Decentralized ledger(s) 117 may be replicated on multiple computing platforms, preferably in multiple different geographical locations. Additionally, individual blocks and/or nodes may be linked together in a manner that prevents tampering, such as using a hash chain and/or digital signatures. In particular, hash values may be generated using fixed-output-length one-way hashing functions that take variable-length input, and may be effectively impossible (or, at least, computationally infeasible) to reverse. As such, a hashing function may provide one-way encryption. By way of non-limiting example, the hashing function may be SHA-256, BLAKE2, SHAKE256, and/or another hashing function. Contents of individual blocks, individual nodes, transactions, and/or assets may be digitally signed in a manner that proves integrity and/or prevents tampering, e.g., by providing authentication, as well as repudiation.

Request receiving component 110 may be configured to receive a request for the psychological profile related to the given subject. The request may be received from an online platform and/or one or more of the online platforms. The online platforms may include the online platform. In some implementations, the stated information, the usage information, and/or other subject information may be requested in the request. The request may be received via a network 118.

Subject authentication component 112 may be configured to authenticate the given subject by obtaining and analyzing authentication information for the given subject. The authentication of the given subject may be responsive to the request and/or receipt of other information. The authentication information for the individual subjects may facilitate verification of the subjects and/or the subject record identifiers.

In some implementations, the request received from the online platform may include the authentication information. In some implementations, the authentication information may indicate whether the subject is logged into the online platform. Thus, analyzing the authentication information may include determining whether the subject is logged into the online platform.

In some implementations, the authentication information may be received from the individual subjects. In some implementations, the authentication information may include subject input entered by the subject via client computing platform 104, biometric information of the subject, and/or other authentication information. The authentication information received may be verified by subject authentication component 112 to determine validity and thus verify the subject and their associated subject information. In some implementations, more than one of the authentication information may be received. In some implementations, one of the authentication information may be received.

The subject input may include a passcode, a password, a pass phrase, and/or other subject input. The biometric information may include a fingerprint, a retinal pattern, a hand scan, a face scan, voice, DNA sample, and/or other biometric information. In some implementations, while the biometric information may facilitate authenticating the given subject, the biometric information may not be associated or included in the subject information. The biometric information may rather facilitate determination of the subject information, or part thereof, to be identified in electronic storage 128. The subject information may identify the individual subjects without personal identification information. The personal identification information may include a name, a gender, an age, a username, an email address, a phone number, the biometric information, and/or other personal identification information.

In some implementations, subject authentication component 112 may be configured to transmit an authentication inquiry to client computing platform 104 associated with the subject. The transmission of the authentication inquiry may be upon receipt of the request. The authentication inquiry may prompt the subject to provide a response. The authentication information may include the response to the authentication inquiry. Analyzing the authentication information may include determining validity of the response. That is, analyzing the authentication information may include determining whether the response is correct or incorrect. By way of non-limiting example, the authentication inquiry may inquire the passcode, the password, the pass phrase, a challenge response to a challenge included in the authentication inquiry, and/or other response. The challenge may include, for example, selecting a particular object or word (or part thereof) from a collection of images, a number of times a particular object or word appears in an image or a video, a riddle, a mathematical question, and/or other challenges. Thus, in some implementations, permission information for the subject information may be determined based on the response to the authentication inquiry being valid.

In some implementations, the verification of the subjects based on the authentication information may facilitate determination of digital wallets associated with the subjects. The digital wallets may correspond to digital addresses tracked and/or recorded on decentralized ledger(s) 117. The digital wallets may indicate the ownership of the non-fungible tokens corresponding to the subject record identifiers, and thus the subject information. As such, the subject information may be identified in electronic storage 128 by the corresponding subject record identifiers.

Permission component 114 may be configured to determine the permission information for the given subject. The permission information may indicate permissions for transmitting the psychological profile and/or other subject information of the given subject to one or more of the online platforms including the online platform. That is, some of the psychological profile and/or other subject information may be permitted for transmission, and some may not. The determination of the permission information may be responsive to authenticating the given subject. In some implementations, the permission information is received from the given subject and stored to electronic storage 128. In some implementations, the permission information is obtained directly from the given subject responsive to authenticating the given subject, and/or other determination techniques.

By way of non-limiting example, the permission information may include timing information related to the one or more online platforms retaining the psychological profile and/or other subject information, particular ones of the psychological parameter values permissioned to be transmitted, restrictions related to utilization of the psychological profile and/or the other subject information by the one or more online platforms, permitted transfer of ownership of the subject information or part thereof, and/or other permission information. In some implementations, the permission information may be altered by the given subject associated with the subject information. In some implementations, the permission information may be altered by the subject or entity that holds the ownership of the non-fungible token that corresponds to the subject information as indicated by decentralized ledger(s) 117. For example, the given subject may hold the ownership of the corresponding non-fungible token, or a company may hold the ownership.

The timing information may include a beginning date and/or time the psychological profile and/or other subject information may be transmitted, an end date and/or time for access to or retainment of the psychological profile and/or other subject information, an amount of time for retainment of the psychological profile and/or other subject information (e.g., one month), time of the day the psychological profile and/or other subject information may be utilized (e.g., 12 AM), and/or other timing information.

In some implementations, some of the psychological parameter values to particular ones of the psychological parameters and/or other subject information may be permitted for transmission. In some implementations, some of the psychological parameters may have a permanent restriction from transmission. In some implementations, some or all of the psychological parameters and/or other subject information may have permissions that may be variable, i.e., may be changed from transmittable to non-transmittable, and vice versa.

The restrictions related to utilization of the psychological profile and/or the other subject information by the one or more online platforms may restrict the one or more online platforms from utilizing the psychological profile and/or the other subject information for research, customization of the one or more online platforms, publication (e.g., in research articles), transmission to other online platforms or entities without permission, duplication, and/or other utilizations.

The permitted transfer of ownership of the subject information, or part thereof may specify one or more entities that may hold the ownership of the subject information. For example, the ownership of the subject information may be represented by the ownership of the corresponding non-fungible token. Thus, in some implementations, one or more specific entities may be permitted to hold the ownership of the corresponding non-fungible token upon the given subject or other entity transferring or relinquishing the ownership. Furthermore, in some implementations, one or more of the entities may be specifically restricted from holding the ownership of the corresponding non-fungible token (e.g., social media platforms, medical institutions). In some implementations, a transfer of the ownership of the corresponding non-fungible token may automatically cause permission component 114 to delete the subject information stored in electronic storage 128. Thus, the subject information is prevented from being owned by anyone or anything else other than the given subject.

In some implementations, determining the permission information may include transmitting a permission inquiry to client computing platform 104 associated with the given subject. Furthermore, an indication may be received, from client computing platform 104 associated with the given subject, that the given subject opts-into, or not, transmission of the psychological profile and/or other subject information to the online platform and/or one or more other ones of the online platforms. The permission inquiry may inquire whether the given subject permits the transmission of the subject information, some of the subject information (e.g., the psychological profile), and/or other information. The permission inquiry may inquire whether the given subject restricts the transmission of particular parts of the subject information (e.g., the usage information, or parts thereof).

In some implementations, the permission information may define entities that may delete and/or alter the subject information. For example, the given subject may be the only entity that may delete and/or alter their subject information. As another example, the given subject and a guardian may be the only entities that may delete and/or alter their subject information.

Profile transmittal component 116 may be configured to transmit the psychological profile and/or other subject information to the online platform and/or one or more of the online platforms. The transmission may be based on the permission information, the subject information or part thereof transmitted, the online platform(s) receiving such, and/or other information.

Supplementation component 120 may be configured to receive additional usage information and/or other information related to the given subject and the online platform. The additional usage information and/or other information may be received from the online platform. The additional usage information may include new values to the usage parameters in relation to the online platform and/or other information that is supplemental to the subject information for the given subject. Supplementation component 120 may be configured to supplement the subject information for the given subject stored in electronic storage 128 with the additional usage information and/or other information received. Thus, the usage information and/or other information related to the given subject may expand as the given subject interacts with more of the online platforms. In some implementations, the psychological profile and/or other subject information may not be transmitted unless the online platform permits transmission of the additional usage information that the online platform collects to system 100. In some implementations, such permission from the online platform may be included in the request from the online platform.

In some implementations, supplementing the subject information may include updating the psychological parameter values for the psychological parameters and/or the values to the various parameters included in the subject information based on the additional usage information and/or the subject information received, collectively, subsequent to the supplementation. For example, in some implementations, supplementing the subject information may include updating overall values to the usage parameters. The overall values to the usage parameters may consider all values to the usage parameters (e.g., an average) in relation to a plurality of the online platforms the given subject has interacted with.

In some implementations, adjustment effectuation component 122 may be configured to effectuate adjustments to a digital environment provided by the online platform for the subject based on the usage information and/or other information. By way of non-limiting example, the adjustments may include adjusting which application are included in the digital environment, the topography, the content provided, access timing information, and/or other information related to the digital environment as described in co-pending U.S. Application Serial No. 17/236,216 entitled “SYSTEMS AND METHODS TO ADAPT A DIGITAL APPLICATION ENVIRONMENT BASED ON PSYCHOLOGICAL ATTRIBUTES OF INDIVIDUAL USERS”, Attorney Docket No. 01TT-064008, the disclosure of which is incorporated by reference in its entirety herein. The access timing information may refer to when the given subject is provided access to the digital environment. The access timing information may include a start time of day, an end time of day, a start date and time, an end date and time, a collective duration over a period of time (e.g., 5 hours a week), the period of time, and/or other access timing information.

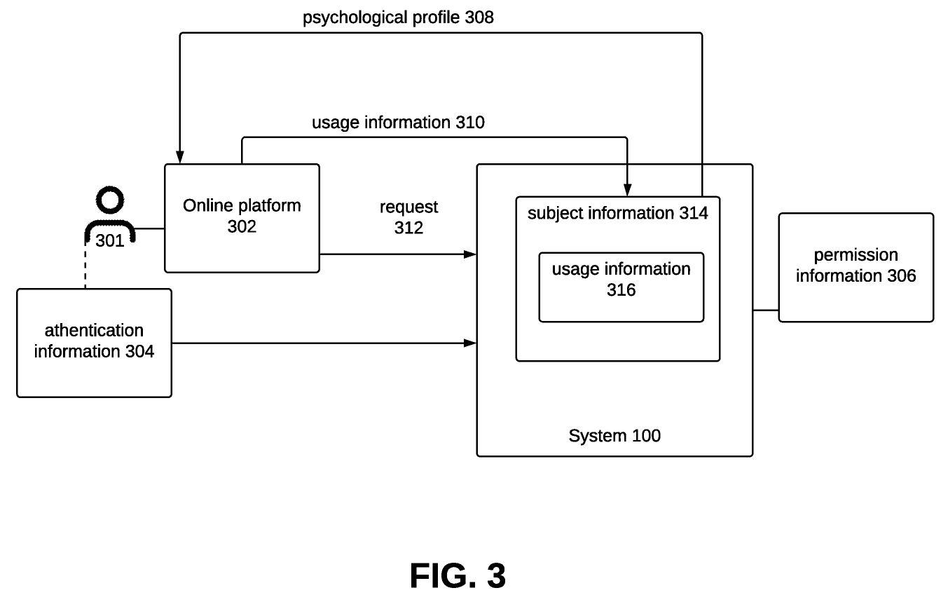

FIG. 3 illustrates a subject 301 who may utilize an online platform 302. Online platform 302 may therefore send a request 312 to system 100 (as described herein in FIG. 1). System 100, responsive to request 312, may receive and analyze authentication information 304 for subject 301. Request 312 may request psychological profile 308 for subject 301. Upon verifying subject 301 by analyzing authentication information 304, system 100 may transmit psychological profile 308 to online platform 302. Based on interactions with online platform 302, online platform 302 may send system 100 usage information 310 that is related to subject 301 using online platform 302. Usage information 316 included in subject information 314 may be supplemented with usage information 310. Thus, in some implementations, psychological profile 308 may be updated based on usage information 310 in addition to subject information 314.

Referring back to FIG. 1, in some implementations, server(s) 102, client computing platform(s) 104, and/or external resources 126 may be operatively linked via one or more electronic communication links. For example, such electronic communication links may be established, at least in part, via network 118 such as the Internet and/or other networks. It will be appreciated that this is not intended to be limiting, and that the scope of this disclosure includes implementations in which server(s) 102, client computing platform(s) 104, and/or external resources 126 may be operatively linked via some other communication media.

A given client computing platform 104 may include one or more processors configured to execute computer program components. The computer program components may be configured to enable an expert or user associated with the given client computing platform 104 to interface with system 100 and/or external resources 126, and/or provide other functionality attributed herein to client computing platform(s) 104. By way of non-limiting example, the given client computing platform 104 may include one or more of a desktop computer, a laptop computer, a handheld computer, a tablet computing platform, a NetBook, a Smartphone, a gaming console, and/or other computing platforms.

External resources 126 may include sources of information outside of system 100, external entities participating with system 100, and/or other resources. In some implementations, some or all of the functionality attributed herein to external resources 126 may be provided by resources included in system 100.

Server(s) 102 may include electronic storage 128, one or more processors 130, and/or other components. Server(s) 102 may include communication lines, or ports to enable the exchange of information with network 118 and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 128 may comprise non-transitory storage media that electronically stores information. The electronic storage media of electronic storage 128 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 128 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 128 may include one or more virtual storage resources (e.g., cloud storage, a virtual private network, and/or other virtual storage resources). Electronic storage 128 may store software algorithms, information determined by processor(s) 130, information received from server(s) 102, information received from client computing platform(s) 104, and/or other information that enables server(s) 102 to function as described herein.

Processor(s) 130 may be configured to provide information processing capabilities in server(s) 102. As such, processor(s) 130 may include one or more of a digital processor, an analog processor, a digital circuit designed to process information, an analog circuit designed to process information, a state machine, and/or other mechanisms for electronically processing information. Although processor(s) 130 is shown in FIG. 1 as a single entity, this is for illustrative purposes only. In some implementations, processor(s) 130 may include a plurality of processing units. These processing units may be physically located within the same device, or processor(s) 130 may represent processing functionality of a plurality of devices operating in coordination. Processor(s) 130 may be configured to execute components 108, 110, 112, 114, 116, 120, and/or 122, and/or other components. Processor(s) 130 may be configured to execute components 108, 110, 112, 114, 116, 120, and/or 122, and/or other components by software; hardware; firmware; some combination of software, hardware, and/or firmware; and/or other mechanisms for configuring processing capabilities on processor(s) 130. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 108, 110, 112, 114, 116, 120, and/or 122 are illustrated in FIG. 1 as being implemented within a single processing unit, in implementations in which processor(s) 130 includes multiple processing units, one or more of components 108, 110, 112, 114, 116, 120, and/or 122 may be implemented remotely from the other components. The description of the functionality provided by the different components 108, 110, 112, 114, 116, 120, and/or 122 described below is for illustrative purposes, and is not intended to be limiting, as any of components 108, 110, 112, 114, 116, 120, and/or 122 may provide more or less functionality than is described. For example, one or more of components 108, 110, 112, 114, 116, 120, and/or 122 may be eliminated, and some or all of its functionality may be provided by other ones of components 108, 110, 112, 114, 116, 120, and/or 122. As another example, processor(s) 130 may be configured to execute one or more additional components that may perform some or all of the functionality attributed below to one of components 108, 110, 112, 114, 116, 120, and/or 122.

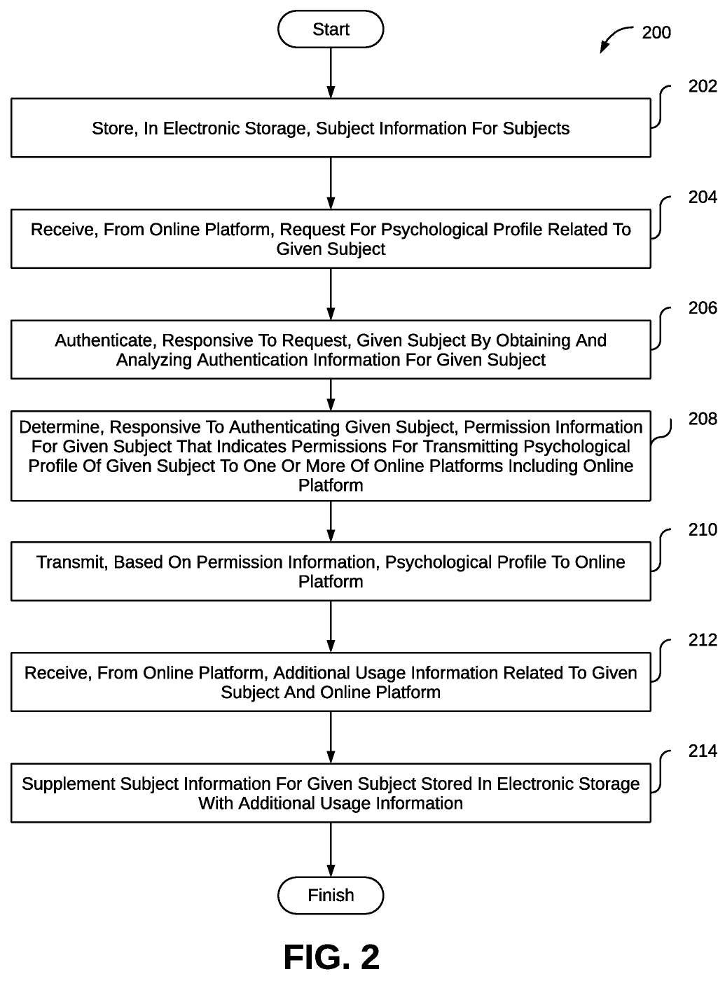

FIG. 2 illustrates a method 200 to facilitate management of online subject information, in accordance with one or more implementations. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

An operation 202 may include storing, in electronic storage, subject information for subjects. The subject information may represent online individualities of the subjects. The subject information for a given subject may include usage information, stated information, and a psychological profile. The usage information may characterize usage of applications within digital environments provided by online platforms. The stated information may include answers to questions presented to the subject that are related to psychological attributes. The psychological profile may include psychological parameter values to psychological parameters derived from the usage information and the stated information. Operation 202 may be performed by a component that is the same as or similar to electronic storage 128, in accordance with one or more implementations.

An operation 204 may include receiving, from an online platform, a request for the psychological profile related to the given subject. Operation 204 may be performed by one or more hardware processors configured by machine-readable instructions including a component that is the same as or similar to request receiving component 110, in accordance with one or more implementations.

An operation 206 may include authenticating, responsive to the request, the given subject by obtaining and analyzing authentication information for the given subject. Operation 206 may be performed by one or more hardware processors configured by machine-readable instructions including a component that is the same as or similar to subject authentication component 112, in accordance with one or more implementations.

An operation 208 may include determining, responsive to authenticating the given subject, permission information for the given subject that indicates permissions for transmitting the psychological profile of the given subject to one or more of the online platforms including the online platform. Operation 208 may be performed by one or more hardware processors configured by machine-readable instructions including a component that is the same as or similar to permission component 114, in accordance with one or more implementations.

An operation 210 may include transmitting, based on the permission information, the psychological profile to the online platform. Operation 210 may be performed by one or more hardware processors configured by machine-readable instructions including a component that is the same as or similar to profile transmittal component 116, in accordance with one or more implementations.

An operation 212 may include receiving, from the online platform, additional usage information related to the given subject and the online platform. Operation 212 may be performed by one or more hardware processors configured by machine-readable instructions including a component that is the same as or similar to supplementation component 120, in accordance with one or more implementations.

An operation 214 may include supplementing the subject information for the given subject stored in the electronic storage with the additional usage information. Operation 214 may be performed by one or more hardware processors configured by machine-readable instructions including a component that is the same as or similar to supplementation component 120, in accordance with one or more implementations.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. A system configured to facilitate management of online subject information, the system comprising:
electronic storage configured to store subject information for subjects, the subject information representing online individualities of the subjects, wherein the subject information for a given subject includes usage information, stated information, and a psychological profile, wherein the usage information characterizes usage of applications within digital application environments provided by online platforms, the stated information includes answers to questions presented to the given subject that are related to psychological attributes, and the psychological profile includes psychological parameter values to psychological parameters derived from the usage information and the stated information; and
one or more processors configured by machine-readable instructions to:
receive, from an online platform, a request for the psychological profile related to the given subject;
authenticate, responsive to the request, the given subject by obtaining and analyzing authentication information for the given subject;
determine, responsive to authenticating the given subject, permission information for the given subject that indicates permissions for transmitting the psychological profile of the given subject to one or more of the online platforms including the online platform;
transmit, based on the permission information, the psychological profile to the online platform;
receive, from the online platform, additional usage information related to the given subject and the online platform; and
supplement the subject information for the given subject stored in the electronic storage with the additional usage information.

2. The system of claim 1, wherein the one or more processors are further
configured by the machine-readable instructions to:
effectuate adjustments to a digital environment provided by the online platform for the subject based on the usage information.

3. The system of claim 1, wherein supplementing the subject information includes updating the psychological parameter values for the psychological parameters based on the additional usage information.

4. The system of claim 1, wherein the authentication information indicates whether the subject is logged into the online platform, wherein analyzing the authentication information includes determining whether the given subject is logged into the online platform.

5. The system of claim 1, wherein the permission information includes timing information related to the one or more online platforms retaining the psychological profile, particular ones of the psychological parameter values permissioned to be transmitted, and/or restrictions related to utilization of the psychological profile by the one or more online platforms.

6. The system of claim 1, wherein the one or more processors are further configured by the machine-readable instructions to:
upon receipt of the request, transmit an authentication inquiry to a client computing platform associated with the given subject, and wherein the authentication information includes a response to the authentication inquiry, wherein analyzing the authentication information includes determining validity of the response, wherein the permission information is determined based on the response being valid.

7. The system of claim 1, wherein the request received from the online platform includes the authentication information.

8. The system of claim 1, wherein determining the permission information includes i) transmitting a permission inquiry to a client computing platform associated with the given subject and ii) receiving, from the client computing platform, an indication that the given subject opts-into transmission of the psychological profile to the online platform.

9. The system of claim 1, wherein the subject information for the subjects are correlated with subject record identifiers.

10. The system of claim 9, wherein the subject record identifiers are associated with non-fungible tokens, wherein a decentralized ledger indicates ownership of the non-fungible tokens.

11. A method to facilitate management of online subject information, the method comprising:
storing, in electronic storage, subject information for subjects, the subject information representing online individualities of the subjects, wherein the subject information for a given subject includes usage information, stated information, and a psychological profile, wherein the usage information characterizes usage of applications within digital application environments provided by online platforms, the stated information includes answers to questions presented to the given subject that are related to psychological attributes, and the psychological profile includes psychological parameter values to psychological parameters derived from the usage information and the stated information;
receiving, from an online platform, a request for the psychological profile related to the given subject;
authenticating, responsive to the request, the given subject by obtaining and analyzing authentication information for the given subject;
determining, responsive to authenticating the given subject, permission information for the given subject that indicates permissions for transmitting the psychological profile of the given subject to one or more of the online platforms including the online platform;
transmitting, based on the permission information, the psychological profile to the online platform;
receiving, from the online platform, additional usage information related to the given subject and the online platform; and
supplementing the subject information for the given subject stored in the electronic storage with the additional usage information.

12. The method of claim 11, further comprising:
effectuating adjustments to a digital environment provided by the online platform for the subject based on the usage information.

13. The method of claim 11, wherein supplementing the subject information includes updating the psychological parameter values for the psychological parameters based on the additional usage information.

14. The method of claim 11, wherein the authentication information indicates whether the subject is logged into the online platform, wherein analyzing the authentication information includes determining whether the given subject is logged into the online platform.

15. The method of claim 11, wherein the permission information includes timing information related to the one or more online platforms retaining the psychological profile, particular ones of the psychological parameter values permissioned to be transmitted, and/or restrictions related to utilization of the psychological profile by the one or more online platforms.

16. The method of claim 11, further comprising:
upon receipt of the request, transmitting an authentication inquiry to a client computing platform associated with the given subject, and wherein the authentication information includes a response to the authentication inquiry, wherein analyzing the authentication information includes determining validity of the response, wherein the permission information is determined based on the response being valid.

17. The method of claim 11, wherein the request received from the online platform includes the authentication information.

18. The method of claim 11, wherein determining the permission information includes i) transmitting a permission inquiry to a client computing platform associated with the given subject and ii) receiving, from the client computing platform, an indication that the given subject opts-into transmission of the psychological profile to the online platform.

19. The method of claim 11, wherein the subject information for the subjects are correlated with subject record identifiers.

20. The method of claim 19, wherein the subject record identifiers are associated with non-fungible tokens, wherein a decentralized ledger indicates ownership of the non-fungible tokens.
